(19) United States
(12) Patent Application Publication
(10) Pub. No.: US 2019/0054159 A1
(43) Pub. Date: Feb. 21, 2019

(54) PHARMACEUTICAL COMPOSITION FOR CANCER TREATMENT, PROCESS FOR ITS PREPARATION, AND ITS USE

(71) Applicant: Spinomics Ltda. - EPP, Porto Alegre, RS (BR)

(72) Inventors: Ricardo Amaral Remer, Rio de Janeiro (BR); Rogério Margis, Porto Alegre (BR); Ana Paula Christoff, Florianópolis (BR)

(73) Assignee: Spinomics Ltda. - EPP, Porto Alegre, RS (BR)

(21) Appl. No.: 15/773,048

(22) PCT Filed: Nov. 1, 2016
(86) PCT No.: PCT/BR2016/050277

§ 371 Date: May 2, 2018

(30) Foreign Application Priority Data
Nov. 6, 2015 (BR) 102015028125-0

(51) Int. Cl. A61K 38/56; A61P 35/00

(52) U.S. Cl. CPC A61K 38/56; A61P 35/00

(57) ABSTRACT

The use of the C-terminal region of one or more phytocystatin(s) for the curative or prophylactic treatment of tumors and/or inhibition of metastases in mammals or other vertebrates. In one embodiment, the pharmaceutical composition of the invention comprises a pharmaceutically acceptable carrier and a polypeptide containing only the C-terminal region of one or more phytocystatin(s). In another embodiment, the composition of the invention comprises a gene construct for the in vivo expression of said polypeptide.

### STATEMENT OF RELATED APPLICATIONS

This application is the US National Phase of International Application No. PCT/BR2016/050277 having an International Filing Date of 1 Nov. 2016, which claims priority on Brazilian Patent Application No. 10 2015 028 125.0 having a filing date of 6 Nov. 2015.

### BACKGROUND OF THE INVENTION

### Technical Field

The industrial creation described herein is in the fields of Biotechnology, Pharmaceutical Sciences and Medicine. More specifically, the present invention relates to the use of a specific group or class of phytocystatins from plant origin for the cancer treatment, as well as a pharmaceutical composition comprising the same.

### Prior Art

Legumains are found primarily located in mammalian lysosomes, but have also been found in association with the extracellular matrix. Endogenously, legumains can be inhibited by cystatins; however, animal cystatins additionally inhibit some cathepsin class proteases, thus lacking activity restricted to a single family of specific cysteine proteinases.

The present invention provides the use of a phytocystatin of plant origin for the cancer treatment, as well as a pharmaceutical composition comprising it for the curative or prophylactic treatment of tumors and/or inhibition of metastases in mammals or other vertebrates. In one embodiment, the composition of the invention comprises a polypeptide comprising only the C-terminal region of one or more phytocystatin(s) with a protein mass greater than 15 kDaltons. In another embodiment, the composition of the invention comprises a gene construct for the in vivo expression of said polypeptide.

Legumains are cysteine proteases belonging to the C13 family (EC 3.4.22.34), also known as asparaginyl endopeptidases (AEP), due to their mechanism of action associated with the recognition and cleavage of asparagine (Asn) sites in target proteins (Abe et al., 1993). These proteases are widely distributed and evolutionarily conserved between plants and animals (Chen et al., 1997; Christoff et al., 2014). In animals, legumains correspond to the only known AEPs and exhibit a structural similarity to caspases, as well as the ability to, in some situations, cleave peptide bonds after aspartic acid residues (Nalfon et al., 1998) (Dall and Brandstetter 2013). Firstly, in the lysosomes, the legumains undergo a catalytic auto-activation process at acidic pH for the removal of propeptides amino and carboxy-terminal (Li et al., 2003) in order to become active.

Although mammalian legumains are located predominantly in lysosomes, they can also be translocated to other cellular compartments such as the nucleus (Haugen et al., 2013), or secreted into tumor microenvironments, interacting with membrane integrins (Liu et al., 2012) (Liu et al., 2003). In the extracellular environment, legumains act by degrading fibronectins (Morita et al., 2007) and activating other proteases such as gelatinase A, important components for the mediation of extracellular matrix degradation (Liu et al., 2003).

Several studies point to the high expression and activity of legumains in mammalian tumor tissues such as breast carcinomas, colon carcinomas, central nervous system neoplasms and prostate tumors, these proteases being poorly expressed in normal tissues (Liu et al. 2003). These data can also be verified in the atlas of human proteins (Uhlen et al., 2010), where the gene expression of legumains can be detected at medium and high levels in several cancer cell lines. Legumains may also be found in macrophages cells associated with the tumor environment, or in primary macrophages, and their activity increases when there is exposure to inflammatory cytokines or tumor cells (Edgington et al., 2013).

In tissues of invasive breast cancer there is a prevalence of legumains expression (Gawenda et al., 2007), also interacting with cancer cell membrane integrins (Liu et al., 2012) and playing the role of key oncogenic enzyme for the maintenance of breast cancer proliferation and metastasis (Lin et al., 2014) (D'Costa et al., 2014).

In types of colorectal cancers, legumains expression is increased and has been positively correlated with carcinoma differentiation (Murthy et al., 2005), in addition to an association with the development of metastases and survival (Haugen et al., 2004). Legumains are also positively correlated with the increased malignancy of ovarian tumors, which have increased cell migration and invasion (Wang et al., 2012), and are also related to the subset of high-risk HPV (Sandberg et al. 2012). More aggressive and invasive prostate tumors were found to have high levels of legumains (Ohno et al., 2013), as well as gastric tumors with metastases (Guo et al., 2013) (Li et al., 2013).

Most of the studies mentioned correlate the increased expression of legumains with a worse prognosis survival of patients, besides suggesting the use of these proteases as a biomarker for the identification of these types of cancer.

More recently, some groups have been studying the use of legumains as biomarkers for the diagnosis of diseases related to these proteases (Yuan et al., 2015). In addition to demonstrating the possibility of using legumains as targets for drug targeting, treatment and disease control (Liu et al., 2015). The development of micelles containing legumains and other drugs has recently been proposed as a method of targeting in the body for drug delivery and local disease control (Lin et al., 2015).

It has already been shown that both auto-activation and legumain activity can be regulated by inhibitors of cysteine proteases such as C, E/M and F cystatin in mammals. However, legumains are not the exclusive target of these cystatins, they also possess a domain capable of inhibiting some cysteine proteases such as papaya (cathepsins) (Abrahamson 1997) (Alvarez-Fernandez et al., 1999) (Cheng et al., 2006). Studies of one of the present inventors (Christoff A P, 2015 Ph.D. Thesis) have confirmed that some plant cystatin (phytocystatins) have an exclusive domain to the C-terminal region of the protein capable of specifically inhibiting plant legumains, in addition to the inhibitory capacity already described at in vitro assays against human legumain (Martinez et al., 2007) and without inhibiting the activity of the papaya/cathepsin type C1A family proteases. More recently, Christoff in his PhD thesis study (Christoff A P, 2015) has demonstrated the relationships between phytocystatins and specifically some of the functions of the C-terminal region of the plant extended phytocystatin in plant biochemistry and/or plant physiology.

In view of this protease/inhibitor relation, a number of studies have evaluated the correlations and interactions between legumains and cystatins in various types of mammalian tumors and found an inhibitory effect of cystatins on tumor cells consistent with the involvement of legumains (Liu et al. 2003). Including data portrayed in the atlas of genetics and cytogenetics in oncology and cytology (Ahmad et al., 2010), where the loss of control of legumains by CST6 (E/M), a human cystatin, implies the progression of cancer. In melanoma cells, tissue invasion is suppressed by the over expression of cystatin E/M, which regulates the activity of legumains (Briggs et al., 2010), also intracellularly and extracellularly in kidney cell lines (Smith et al. 2012). This cystatin is in decreased content in metastatic breast cancer cells and gliomas, and in many cases by epigenetic alterations (Sotiropoulou et al., 1997) (Shridhar et al., 2004) (Zhang et al. 2004) (Ai et al. 2006) (Schagdarsurengin et al., 2007) (Qiu et al., 2008). Thus, cystatins with inhibitory capacity for legumains have been postulated as important candidates for tumor suppressors and metastases in mammals.

Based on these studies of legumain activities (proteases C13), different methodologies have been proposed for the development of cancer tissue biomarkers (Edgington et al. 2013) (Chen et al. 2014) (Jiang et al. 2014) and also for the creation of new drugs that may aid in the treatment of cancer (Sutherland et al. 2006) (Drag and Salvesen 2010) (Scott and Taggart 2010) (Lee and Bogyo 2012) (Liu et al. 2014) (Smith and Astrand 2014). However, none of the studies to which the inventors have had access has disclosed, considered or suggested the use of extended C-terminal regions of phytocystatins for the treatment of cancer in vertebrates, including mammals.

To date, only a few studies have suggested the possibility of using animal cystatins (non-extended, less than 15 kDa) for cancer treatment, but not phytocystatins. However the focus of these studies is on the inhibition of cathepsins B and L (proteases C1A) and their important roles in the invasion of tumor cells in breast cancer (Gianotti et al., 2008). Or further by inhibiting cathepsins in the formation of melanomas, interfering with the process of angiogenesis and preventing the invasion and migration of melanoma (Oliveira et al., 2011). Differently from the invention herein, these previous studies focus on a different subset of phytocystatins (non-extended), whose role is to inhibit a different subset of cysteine proteases belonging to the family CIA.

Thus, a novel and unexpected character of the invention herein is in the use of one or more plant phytocystatins, greater than 15 kDa, whose C-terminal extent has the selective inhibition ability of legumain-like proteases, which belong to the family C13.

The scientific literature restrains the invention and mentioned above includes:
Abe Y, Shirane K, Yokosawa H, et al. (1993) Asparaginyl Endopeptidase of Jack Bean Seeds. Biol Chem 268:3525-3529.
Abrahamson M (1997) Cystatin E is a Novel Human Cysteine Proteinase Inhibitor with Structural Resemblance to Family 2Cystatins. J Biol Chem 272:10853-10858. doi: 10.1074/jbc.272.16.10853.
Ai L, Kim W-J, Kim T-Y, et al. (2006) Epigenetic silencing of the tumor suppressor cystatin M occurs during breast cancer progression. Cancer Res 66:7899-909. doi: 10.1158/0008-5472. CAN-06-0576.
Ahmad M, Arsaban M, Delabrousse H, Labarussias M, Berre V, Malo a., Dessen P, Huret J L. 2010. Atlas of Genetics and Cytogenetics in Oncology and Haematology: an Internet information source with free access. Oncologie 12, 685-686.
Alvarez-Fernandez M, Barrett a J, Gerhartz B, et al. (1999) Inhibition of mammalian legumain by some cystatins is due to a novel second reactive site. J Biol Chem 274:19195-203.
Briggs J J, Haugen M H, Johansen H T, et al. (2010) Cystatin E/M suppresses legumain activity and invasion of human melanoma. BMC Cancer 10:17. doi: 10.1186/1471-2407-10-17.
Chen J, Dando P M, Rawlings N D, et al. (1997) Cloning, Isolation, and Characterization of Mammalian Legumain, an Asparaginyl Endopeptidase. J Biol Chem 272:8090-8098.
Chen Y, Wu S, Chen C, Tzou S (2014) Peptide-based MRI contrast agent and near-infrared fluorescent probe for intratumor legumain detection. Biomaterials 35:304-315.
Cheng T, Hitomi K, van Vlijmen-Willems I M J J, et al. (2006) Cystatin M/E is a high affinity inhibitor of cathepsin V and cathepsin L by a reactive site that is distinct from the legumain-binding site. A novel clue for the role of cystatin M/E in epidermal cornification. J Biol Chem 281:15893-9. doi: 10.1074/jbc. M600694200.
Christoff A P, Margis R (2014) The diversity of rice phytocystatins. Mol Genet Genomics 1-10. doi: 10.1007/s00438-014-0892-7.
Christoff A P, Turchetto-Zolet A C, Margis R (2014) Uncovering legumain genes in rice. Plant Sci 215-216:100-9. doi: 10.1016/j.plantsci.2013.11.005.
Christoff, A P. Diversidade de fitocistatinas em arroz e suas proteinases cisteinicas-alvo, com enfoque em fitocistatinas carboxi-estendidas e inibição de legumainas. 14 Sep. 2015. 141 páginas. Tese de Doutorado. Universidade Federal do Rio Grande do Sul, Departamento de Genética.
D'Costa Z, Higgins C, Ong C, Irwin G (2014) TBX2 represses CST6 resulting in uncontrolled legumain activity to sustain breast cancer proliferation: a novel cancer-selective target pathway with therapeutic. Oncotarget 5:
Dall E, Brandstetter H (2013) Mechanistic and structural studies on legumain explain its zymogenicity, distinct activation pathways, and regulation. Proc Natl Acad Sci USA 110:10940-5. doi: 10.1073/pnas.1300686110.
Drag M, Salvesen G S (2010) Emerging principles in protease-based drug discovery. Nat Rev Drug Discov 9:690-701. doi: 10.1038/nrd3053.
Edgington L E, Verdoes M, Ortega A, et al. (2013) Functional imaging of legumain in cancer using a new quenched activity-based probe. J Am Chem Soc 135:174-82. doi: 10.1021/ja307083b.
Gawenda J, Traub F, Lück H J, et al. (2007) Legumain expression as a prognostic factor in breast cancer patients. Breast Cancer Res Treat 102:1-6. doi: 10.1007/s10549-006-9311-z.
Gianotti A, Sommer C a., Carmona A K, Henrique-Silva F. 2008. Inhibitory effect of the sugarcane cystatin CaneCPI-4 on cathepsins B and L and human breast cancer cell invasion. Biological Chemistry 389, 447-453.
Guo P, Zhu Z, Sun Z, et al. (2013) Expression of legumain correlates with prognosis and metastasis in gastric carcinoma. PLoS One 8:e73090. doi: 10.1371/journal.pone.0073090.
Halfon S, Patel S, Vega F, et al. (1998) Autocatalytic activation of human legumain at aspartic acid residues. FEBS Lett 438:114-118.
Haugen M H, Boye K, Nesland J M, et al. (2014) High expression of the cysteine proteinase legumain in colorectal cancer—Implications for therapeutic targeting. Eur J Cancer. doi: 10.1016/j.ejca.2014.10.020.
Haugen M H, Johansen H T, Pettersen S J, et al. (2013) Nuclear legumain activity in colorectal cancer. PLoS One 8:e52980. doi: 10.1371/journal.pone.0052980.
Jiang Y, Lu J, Wang Y, et al. (2014) Molecular-Dynamics-Simulation-Driven Design of a Protease-Responsive Probe for In Vivo Tumor Imaging. Adv Mater 1-5. doi: 10.1002/adma.201403547.
Lee J, Bogyo M (2012) Synthesis and evaluation of aza-peptidyl inhibitors of the lysosomal asparaginyl endopeptidase, legumain. Bioorg Med Chem Lett 22:1340-3. doi: 10.1016/j.bmc1.2011.12.079.
Li D N, Matthews S P, Antoniou A N, et al. (2003) Multistep autoactivation of asparaginyl endopeptidase in vitro and in vivo. J Biol Chem 278:38980-90. doi: 10.1074/jbc.M305930200.
Li N, Liu Q, Su Q, et al. (2013) Effects of legumain as a potential prognostic factor on gastric cancers. Med Oncol 30:621. doi: 10.1007/s12032-013-0621-9.
Lin Y, Qiu Y, Xu C, et al. (2014) Functional role of asparaginyl endopeptidase ubiquitination by TRAF6 in tumor invasion and metastasis. J Natl Cancer Inst 106:dju012. doi: 10.1093/jnci/dju012.
Lin S, Deng F, Huang P, Li L, Wang L, Li Q, Chen L, Chen H, Nan K. 2015. A novel legumain protease-activated micelle cargo enhances anticancer activity and cellular internalization of doxorubicin. J. Mater. Chem. B 3, 6001-60.
Liu C, Sun C, Huang H, et al. (2003) Overexpression of legumain in tumors is significant for invasion/metastasis and a candidate enzymatic target for prodrug therapy. Cancer Res 2957-2964.
Liu Y, Bajjuri K M, Liu C, Sinha S C (2012) Targeting cell surface alpha(v)beta(3) integrin increases therapeutic efficacies of a legumain protease-activated auristatin prodrug. Mol Pharm 9:168-75. doi: 10.1021/mp200434n.
Liu Z, Xiong M, Gong J, et al. (2014) Legumain protease-activated TAT-liposome cargo for targeting tumours and their microenvironment. Nat Commun 5:4280. doi: 10.1038/ncomms5280.
Liu Y, Goswami R K, Liu C, Sinha S C. 2015. Chemically Programmed Bispecific Antibody Targeting Legumain Protease and αvβ3 Integrin Mediates Strong Antitumor Effects. Molecular Pharmaceutics, 150609092733002.
Margis R, Reis E M, Villeret V (1998) Structural and phylogenetic relationships among plant and animal cystatins. Arch Biochem Biophys 359:24-30. doi: 10.1006/abbi.1998.0875.
Martinez M, Diaz-Mendoza M, Carrillo L, Diaz I (2007) Carboxy terminal extended phytocystatins are bifunctional inhibitors of papain and legumain cysteine proteinases. FEBS Lett 581:2914-8. doi: 10.1016/j.febslet.2007.05.042.
Morita Y, Araki H, Sugimoto T, et al. (2007) Legumain/asparaginyl endopeptidase controls extracellular matrix remodeling through the degradation of fibronectin in mouse renal proximal tubular cells. FEBS Lett 581:1417-24. doi: 10.1016/j.febslet.2007.02.064.
Murthy R, Arbman G, Gao J (2005) Legumain expression in relation to clinicopathologic and biological variables in colorectal cancer. Clin cancer 11:2293-2299.
Ohno Y, Nakashima J, Izumi M, et al. (2013) Association of legumain expression pattern with prostate cancer invasiveness and aggressiveness. World J Urol 31:359-64. doi: 10.1007/s00345-012-0977-z.
Oliveira J P, Magliarelli H F, Pereira F V., et al. 2011. Sugarcane cystatin caneCPI-4 inhibits melanoma growth by angiogenesis disruption. Journal of Cancer Science and Therapy 3, 161-167.
Qiu J, Ai L, Ramachandran C, et al. (2008) Invasion suppressor cystatin E/M (CST6): high-level cell type-specific expression in normal brain and epigenetic silencing in gliomas. Lab Invest 88:910-25. doi: 10.1038/labinvest.2008.66.
Sandberg A, Lindell G, Källström B N, et al. (2012) Tumor proteomics by multivariate analysis on individual pathway data for characterization of vulvar cancer phenotypes. Mol Cell Proteomics 11:M112.016998. doi: 10.1074/mcp.M112.016998.
Schagdarsurengin U, Pfeifer G P, Dammann R (2007) Frequent epigenetic inactivation of cystatin M in breast carcinoma. Oncogene 26:3089-94. doi: 10.1038/sj.onc.1210107.
Scott C J, Taggart C C (2010) Biologic protease inhibitors as novel therapeutic agents. Biochimie 92:1681-8. doi: 10.1016/j.biochi.2010.03.010.
Shridhar R, Zhang J, Song J, et al. (2004) Cystatin M suppresses the malignant phenotype of human MDA-MB-4355 cells. Oncogene 23:2206-15. doi: 10.1038/sj.onc.1207340.
Smith R, Åstrand O (2014) Synthesis of a novel legumain-cleavable colchicine prodrug with cell-specific toxicity. Bioorganic Med chemestry 22:3309-3315.
Smith R, Johansen H T, Nilsen H, et al. (2012) Intra- and extracellular regulation of activity and processing of legumain by cystatin E/M. Biochimie 94:2590-9. doi: 10.1016/j.biochi.2012.07.026.
Sotiropoulou G, Anisowicz a., Sager R (1997) Identification, Cloning, and Characterization of Cystatin M, a Novel Cysteine Proteinase Inhibitor, Down-regulated in Breast Cancer. J Biol Chem 272:903-910. doi: 10.1074/jbc.272.2.903.
Sutherland M S K, Sanderson R J, Gordon K a, et al. (2006) Lysosomal trafficking and cysteine protease metabolism confer target-specific cytotoxicity by peptide-linked anti-CD30-auristatin conjugates. J Biol Chem 281:10540-7. doi: 10.1074/jbc. M510026200.
Uhlen M, Oksvold P, Fagerberg L, et al 2010. Towards a knowledge-based Human Protein Atlas. Nature biotechnology 28, 1248-1250.
Wang L, Chen S, Zhang M, et al. (2012) Legumain: a biomarker for diagnosis and prognosis of human ovarian cancer. J Cell Biochem 113:2679-86. doi: 10.1002/jcb.24143.
Zhang J, Shridhar R, Dai Q, et al. (2004) Cystatin: a novel candidate tumor Suppressor gene for breast cancer. Cancer Res 6957-6964.
Yuan Y, Ge S, Sun H, Dong X, Zhao H, An L, Zhang J, Wang J, Hu B, Liang G. 2015. Intracellular Self-Assembly and Disassembly of 19 F Nanoparticles Confer Respective ‘Off’ and ‘On’ 19 F NMR/MRI Signals for Legumain Activity Detection in Zebrafish. ACS Nano 9, 5117-5124.

The patent literature relating to the present invention includes the following documents:

WO 2006/066358, filed by Sugar Industry Innovation PTU ltd. and entitled “Vacuole Targeting Peptide and Nucleic Acid”, discloses a nucleotide sequence and a peptide sequence that directs the vacuole. Gene constructs and the expression of chimeric proteins in plants, notably monocotyledons, such as cereals and sugar cane, are revealed.

WO 02 094980, entitled “Phytocystatin”, discloses a cystatin of Vigna unguiculata and its pharmaceutical, cosmetic and agronomic uses, in particular for transgenic plants—to produce plants resistant to certain stresses, such as desiccation.

WO 0070071, entitled “Senescence-related Promoters”, discloses transcriptional initiation regions and promoters isolated from corn and characterized. Such regions are capable of modifying gene expression during plant senescence. The revealed matter, therefore, relates to the use of these regions in gene constructions that modify the gene expression of the plant.

The present invention differs from all such documents for various technical reasons. For example, none of such documents disclose or even suggest the approach of treating mammalian or other vertebrate cancers with polypeptides provided exclusively with the carboxy-terminal region of plant phytocystatins. Nor do they disclose or suggest its use in compositions for the preventive, curative or prophylactic treatment of cancer and/or inhibition of metastases.

Based on the patent and non-patent literature that the inventors had access to, it is clear that there is a need to search for new alternatives solutions to existing ones to overcome the limitations of the prior art. The present patent application discloses solutions to these problems.

As can be seen from the literature researched, no documents have been found anticipating or suggesting the teachings of the present invention which, for the inventors, has novelty and inventive activity towards the prior art.

### BRIEF SUMMARY OF THE INVENTION

The present invention has as a common inventive concept for its various objects the cancer treatment of mammalian or other vertebrate through the use of a polypeptide comprising one or more sequences having similarity equal to or greater than 70% to SEQ ID No: 6, or one or more sequences having identity equal to or greater than 70% with SEQ ID No: 6.

Said polypeptide resembles phytocystatin(s), but comprises only the selective ligand carboxy-terminal domain of legumain, a type of protease, modulating its activity and/or exclusive inhibitor of legumain(s), unlike known animal cystatins, which also inhibit other targets. In the present invention, it is surprisingly shown that said polypeptide is potentially useful for preparing a composition for cancer treatment—by acting as suppressor of tumor and/or metastasis formation.

One of the objects of the invention is a pharmaceutical composition for the cancer treatment of mammalian and/or other vertebrate, the composition comprising:
a pharmaceutically acceptable carrier; and
one or more polypeptides comprising one or more sequences having an similarity equal to or greater 70% to SEQ ID No: 6, or one or more sequences having an identity equal to or greater 70% to SEQ ID No: 6; or
one or more gene constructs useful for the expression, in mammals and/or other vertebrates, of one or more polypeptides as defined above.

In one embodiment, the composition of the invention comprises a polypeptide comprising an arrangement of two or more fused, repeated, or sequenced polypeptide sequences of different carboxy-terminal domains of phytocystatins.

In one embodiment, the composition of the invention comprises a gene construct containing an arrangement of two or more nucleotide sequences encoding said polypeptides fused in phase, repeated or in sequence of equal or different nucleotide sequences having a similarity of greater than or equal to 70% to SEQ ID No: 5, or identity greater than or equal to 70% to SEQ ID No: 5, for administration of the gene construct and expression of the polypeptide(s) directly into the mammal/vertebrate organism.

The pharmaceutical composition of the invention is in oral, sublingual, injectable, transdermal, topical (cream, gel or emulsion), inhalable dosage form and/or as a suppository.

In one embodiment, the pharmaceutical composition of the invention comprises a gene construct or expression vector comprising:
one or more nucleotide sequences having similarity equal to or greater than 70% to SEQ ID No: 5, or one or more nucleotide sequences having identity equal to or greater than 70% to SEQ ID No: 5;
a functional promoter in mammals and/or other vertebrates, bonded to the encoding nucleotide sequence described above; and
a nucleotide sequence selected from: a transcription terminator nucleotide sequence; a selection marker; a secretion signal sequence; a sequence facilitating export; a nucleotide sequence encoding another protease inhibitory polypeptide sequence; combinations thereof, or yet a plasmid comprising such sequences,
   wherein any of the nucleotide sequences defined above is heterologous.

Another object of the invention is a gene construct or expression vector for the expression of one or more polypeptides useful for the cancer treatment of mammalian or other vertebrate, said gene construct comprising:
one or more nucleotide sequences with similarity equal to or greater than 70% to SEQ ID No: 5, or one or more nucleotide sequences with identity equal to or greater than 70% to SEQ ID No: 5;
a functional promoter in the target organism, linked to the encoding nucleotide sequence described above; and
a nucleotide sequence selected from: a transcription terminator nucleotide sequence; a selection marker; a secretion signal sequence; a sequence facilitating export; a nucleotide sequence encoding another protease inhibitory polypeptide sequence; combinations thereof, or yet a plasmid comprising such sequences,
   wherein any of the nucleotide sequences defined above is heterologous.

It is another object of the invention the use the polypeptide described above or the gene construct described above for the preparation of a cancer drug.

Another object of the invention is a preparation process of a cancer drug, said process comprising: a step of mixing a polypeptide as described above, or a gene construct as described above; and a pharmaceutically acceptable carrier.

These and other objects of the invention will be readily appreciated by those skilled in the art and by companies having an interest in field and will be described in sufficient detail for their reproduction in the following description.

### BRIEF DESCRIPTION OF THE DRAWINGS

FIG. 1 shows schematic representations of embodiments of the invention: in A) there is shown an artificial/unnatural polypeptide sequence of SEQ ID No: 6, comprising a carboxy-terminal region derived from rice phytocystatin; in B) there is shown a sequence of another single recombinant polypeptide resulting respectively from the combination of two carboxy-terminal regions of papaya cystatin, followed by a carboxy-terminal region of a corn cystatin and a carboxy-terminal region of a rice cystatin.

FIG. 2 shows a schematic representation of gene construct embodiments of the present invention, indicated: in A) 21, a plasmid pCAMBIA, which enables expression in both plants and bacteria for the expression of a polypeptide encoded by SEQ ID No. 6; 22, an origin of replication; 23, a promoter operably linked to the encoding sequence; 24, the coding sequence SEQ ID No: 5; and 25, a reporter gene (GFP), the transcription terminator region; in B) there is shown the schematic representation of the plasmid pRSFDuet™-1, which provides for the co-expression of two encoding sequences of interest. This vector contains two multiple cloning sites (MCS), each preceded by a promoter T7lac and a ribosome binding site (rbs).

FIG. 3 shows a graph indicative of the number of cells U343 after 5 days of treatment with one embodiment of the invention comprising a recombinant rice phytocystatin (O. sativa) containing only the carboxy-terminal portion. On the axis “y”, the total number of cells is represented and, on the axis “x” the results of each tested condition are represented, respectively: control cell U343 (without administration of the composition of the invention); and cells U343 that received treatments of 1, 10 and 50 micromolar of said polypeptide.

FIG. 4 shows a graph indicative of the number of cells A172 after 5 days of treatment with the composition indicated in the description of FIG. 3. On the axis “y” the total number of cells is represented and, on the axis “x” are represented the results of each condition tested, respectively: control cells A172 (without administration of the composition of the invention); and cells A172 that received treatments of 1, 10 and 50 micromolar of said polypeptide.

FIG. 5 shows a graph indicative of the number of cells C6 after 5 days of treatment with the composition indicated in the description of FIG. 3. On the axis “y” the total number of cells is represented and, on the axis “x” are represented the results of each condition tested, respectively: control cells C6 (without administration of the composition of the invention); and cells C6 that received treatments of 1, 10 and 50 micromolar treatments of said polypeptide.

FIG. 6 shows a graph indicative of the number of cells U138 after 5 days of treatment with the composition indicated in the description of FIG. 3. On the axis “y” the total number of cells is represented and, on the axis “x” are represented the results of each condition tested, respectively: control cells U138 (without administration of the composition of the invention); and cells U138 that received treatments of 1, 10 and 50 micromolar of said polypeptide.

FIG. 7 shows an illustrative graph of cumulative cell proliferation over time for glioma cells U343 subjected to treatment with the composition indicated in the description of FIG. 3. On the axis “y” cumulative cell duplication is represented and, on the axis “x” represents the time in days. They are shown: in A, the proliferation/control curve, that is, without the administration of the active; in B, C and D the curves relative to the administration of said polypeptide, respectively at concentrations 1, 10 and 50 micromolar.

FIG. 8 shows an illustrative graph of cumulative cell proliferation over time for glioma cells A172 subjected to treatment with the composition indicated in the description of FIG. 3. On the axis “y” cumulative cell duplication is represented and, on the axis “x” the time in days is represented. They are shown: in A, the proliferation/control curve, that is, without the administration of the active; in B, C and D the curves relative to the administration of said polypeptide, respectively at concentrations 1, 10 and 50 micromolar.

FIG. 9 shows an illustrative graph of cumulative cell proliferation over time for glioma cells C6 subjected to treatment with the composition indicated in the description of FIG. 3. On the axis “y”, cumulative cell duplication is represented, and, on the axis “x” is represented the time in days. They are shown: in A, the proliferation/control curve, that is, without the administration of the active; in B, C and D the curves relative to the administration of said polypeptide, respectively at concentrations 1, 10 and 50 micromolar.

FIG. 10 shows an illustrative graph of cumulative cell proliferation over time for glioma cells U138 subjected to treatment with the composition indicated in the description of FIG. 3. On the axis “y”, cumulative cell duplication is represented, and, on the axis “x” is represented the time in days. They are shown: in A, the proliferation/control curve, that is, without the administration of the active; in B, C and D the curves relative to the administration of said polypeptide, respectively at concentrations 1, 10 and 50 micromolar.

FIG. 11 shows a schematic representation of the gene sequence data of rice phytocystatin with carboxy-extended terminal. The representation of the complete nucleotide sequence of the locus in rice chromosome 1, indicated: the white regions correspond to the introns and the shaded regions correspond to the exons.

### DETAILED DESCRIPTION OF PREFERRED EMBODIMENTS

Legumains are found primarily located in mammalian lysosomes, but have also been found in association with the extracellular matrix. Endogenously, legumains can be inhibited by cystatin; however, animal cystatins additionally inhibit some cathepsin class proteases, thus lacking a specific activity. The present invention provides a composition for the legumains selective binding and/or inhibition of mammalian and/or other vertebrate.

The present invention has as an inventive concept common to its various objects the use, for preparing a medicament for cancer treatment of mammalian or other vertebrate, of a polypeptide comprising one or more sequences having similarity equal to or greater than 70% with SEQ ID No: 6, or one or more sequences with identity equal to or greater than 70% with SEQ ID No: 6.

Said polypeptide resembles phytocystatin(s), but comprises only selective ligand carboxy-terminal domain of legumain, a type of protease, modulating its activity and/or exclusive inhibitor of legumain(s), unlike known animal cystatins, which also inhibit other targets. In the present invention, it is surprisingly shown that said polypeptide is useful for cancer treatment of mammalian and/or other vertebrate by acting as suppressor of tumor and/or metastasis formation.

Cancer cannot be considered a single disease in its etiology, there being more than 100 different types named by the organ or cell type in which the disease begins. Characterized by a disordered growth of cells, cancer cells can invade other tissues, spreading through the body through metastases. According to data collected and released by the National Institutes of Health (NIH), WHO (World Health Organization) and INCA (Instituto Nacional do Cancer), in 2012 there were 14.1 million new cases of cancer and 8.2 million deaths in the world. Prospects for 2030 include 21.4 million new cases and 13.2 million cancer deaths globally. Given these high numbers, cancer is a major public health problem—deeply affecting at least 1 million people diagnosed per year.

Cancer treatments are advancing, saving lives and extending the survival rate of people with various types of cancer. Although most causes of cancer are known to be multi-factorial and range among subjects, some molecular pathways, which are already known, are similar in some types of cancer. One of these pathways relates to the altered and increased activity of cysteine proteases of the legumains type. Independently, this increased activity has already been detected in different types of cancer including breast, colon, lung, prostate, ovary, and kidneys, among others. In most of these studies, tumors presenting a greater activity of legumains are associated with a high level of metastases and a worse prognosis of survival.

Thus, recent works have been attempting to develop drugs that specifically aim legumains. In general, there is a greater focus for synthetic prodrug research and some studies focus on the use of animal cystatin as legumain inhibitors. However, animal cystatin, although currently under intense study, provides low specificity for the inhibition of animal legumains. In this way, the research and development of other molecular entities continues to be an important demand for the development of new strategies for the cancer treatment.

The wide diversity of cancer types in which an increase of legumain-like proteases in association with aggressiveness and metastasis formation is found justifies efforts and investments in the development of effective inhibitors of low or no side effects and with therapeutic potential. In this context, the present invention meets, among others, the objectives of the Federal Government's incentive (Decree No. 6,041, February 2007) for the generation and national production of new biomolecules and/or recombinant proteins of therapeutic interest.

The present invention provides solutions to the problems of the prior art by providing a candidate useful for cancer treatment of mammalian or vertebrate through a polypeptide of plant origin.

For purposes of the present invention the following definitions are used:

Recombinant or modified polypeptide: In the context of the present patent application, “non-natural polypeptide, or recombinant polypeptide, or modified polypeptide” is to be understood as a non-naturally occurring polypeptide.

Target organism: In the context of the present patent application, the term “target organism” means those organisms which receive the gene construct of the invention, so that the gene expression occurs in the target organism. Said target organism includes, but is not limited to, bacteria, yeasts and other fungi, plants or other plant cells, animal, mammals and/or other vertebrates cells.

Pharmaceutical composition: In the context of the present patent application, “pharmaceutical composition” is to be understood as any and all compositions containing an active ingredient, for prophylactic, palliative and/or curative purposes, acting in a manner to maintain and/or recovering homeostasis, and may be administered topically, parenterally, enterally, intrathecally, orally, intravenously, intranasally, intravitreally and intramuscularly, intracerebrally, intracerebroventricularly and intraocularly in its administration and/or formulation.

“Pharmaceutically acceptable formulation or carrier”: In the context of the present patent application, a “pharmaceutically acceptable formulation or carrier” is understood to mean a formulation containing pharmaceutically acceptable excipients and carriers well known to those skilled in the art, as it is the development of convenient doses and treatments for use in particular compositions which may be described in a variety of treatment regimens, including oral, topical, parenteral, intravenous, intranasal, intravitreal and intramuscular, intracerebral, intracerebroventricular and intraocular and its administration and/or formulation.

Cellular disorder: In the context of the present patent application, “cellular disorders” is to be understood as any cellular disorder which alters the normal physiological functions of a cell or tissue, especially mammals or other vertebrates. In the context of the present patent application the term encompasses any biochemical or molecular change resulting in or is associated with altered expression and/or activity of legumain(s) in cells or tissues, including neoplasms.

The present invention contributes first, to scientific knowledge about the activity of plant cysteine protease inhibitors for cancer treatment of mammalian or other vertebrate. Throughout the evolution of organisms, several genes and proteins remained with related functions between the two realms. However, although it is known that legumains are involved in the process of developing tumors and metastases and that cystatins have the ability to inhibit the activity of these proteases, to the best of the inventors' knowledge there was at present no available report of the approach to use and effectively to test only the C-terminal region of plant phytocystatins for cancer treatment in mammalians. Perhaps the fact that plant inhibitors have progressively acquired the specific inhibitory property only for legumains explains this gap. The present invention provides an innovative approach by extending these types of analyzes across the realms of organisms, providing for the identification of biomolecules with therapeutic potentials. In addition, plant systems have several advantages over others in the production of new drugs because they require much less effort and investments in preclinical testing than conventional systems.

Among other objects, the present invention provides a pharmaceutical composition for cancer treatment of mammalian and/or other vertebrate, the composition comprising:
a pharmaceutically acceptable carrier; and
one or more polypeptides comprising one or more polypeptide sequences having a similarity equal to or greater than 70% with SEQ ID No: 6, or one or more sequences having identity equal to or greater than 70% with SEQ ID No: 6.

The following examples illustrate some of the embodiments of the invention without, however, limiting the scope thereof.

### Example 1—a Composition for Cancer Treatment in Mammals and/or Other Vertebrates—Comprising Recombinant Polypeptide

Carboxy-extended phytocystatins gene regions of previously known plants were selected for evaluation of the functionality of the resulting recombinant polypeptides. The carboxy-extended phytocystatins genes of the plants were obtained from: arabidopsis (Arabidopsis thaliana), papaya (Carica papaya), soybean (Glycine max), rice (Oryza sativa), corn (Zea mays) and eucalyptus (Eucaliptus grandis). The choice of the genes from these species was based on previously presented phylogenies (Christoff and Margis 2014; Christoff 2015), in order to contemplate the inclusion of the species that represent a good sampling of the diversity of phytocystatins in plants. The encoding sequences of the C-terminal regions of phytocystatins from different plants were inserted into the target vectors using the restriction enzyme system of each vector and subsequent ligation with DNA ligase.

In the composition of the invention, the polypeptide is selected from among different variants, among which has higher inhibitory activity (Ki) against mammalian or human legumain. The element common to the different embodiments and composition of the invention is a polypeptide comprising a specific region of a carboxy-extended phytocystatin, independently of the parent plant or being a synthetic polypeptide. In this embodiment, the composition of the invention comprises a recombinant phytocystatin extract from rice (O. sativa) containing only the carboxy terminal portion, as indicated in SEQ ID No: 6 and FIG. 1, part A).

### Example 2—Gene Construct

The gene construct of the invention is useful for the expression of one or more polypeptides useful for cancer treatment of mammalian or other vertebrate. Said gene construct comprises:
one or more nucleotide sequences with similarity equal to or greater than 70% with SEQ ID No: 5, or one or more nucleotide sequences with identity equal to or greater than 70% with SEQ ID No: 5;
a functional promoter in the target organism, linked to the encoding nucleotide sequence described above; and
a nucleotide sequence selected from: a transcription terminator nucleotide sequence; a selection marker; a secretion signal sequence; a sequence facilitating export; a nucleotide sequence encoding another protease inhibitory polypeptide sequence; combinations thereof, or yet a plasmid comprising such sequences,
   wherein any of the nucleotide sequences defined above being heterologous.

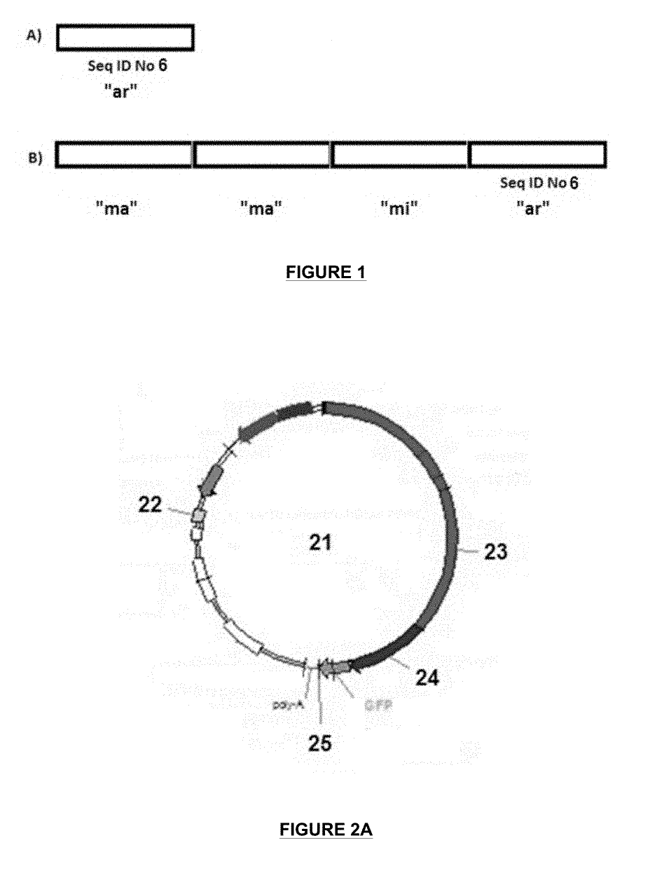

In this embodiment, schematically illustrated in FIG. 2A, the gene construct of the invention comprises as a polypeptide encoding sequence SEQ ID No.5, as shown in Example 1.

It should be noted that each expression system provides advantages and disadvantages, with the choice of the most efficient system for the expression of the biomolecules being in each case a matter of weighing the amount of protein recovered and/or the degree of purity thereof. Similarly, the gene construct of the invention may be directed to expression of the recombinant polypeptide of the invention in bacteria, yeast, plants, for subsequent administration, or still directly to mammals or other vertebrates.

### Example 3—Composition Comprising Recombinant Polypeptide in Arrangement of Fused Sequences

In this embodiment, the polypeptide comprises a fused sequence arrangement with four carboxy-terminal domains of phytocystatins, two of which are repeat papaya followed by one of corn and another of rice, as schematically illustrated in FIG. 1, part B). Said sequence arrangement is also selective ligand of legumain(s), being useful in the modulation and/or inhibition of its activity. The preparation of this composition is made according to the process of the invention, which comprises a mixing step of:
one or more polypeptides comprising one or more sequences having similarity equal to or greater than 70% with SEQ ID No: 6, or one or more sequences having identity equal to or greater than 70% with SEQ ID No: 6; or
one or more gene constructs useful for the expression in mammals and/or other vertebrates of one or more of said polypeptides; and
a pharmaceutically acceptable carrier.

### Example 4—Tests on Mammalian Cancer Lines

The composition of the invention has surprisingly been shown to provide curative or prophylactic treatment of a variety of cell disorders, notably cancer. The composition of this embodiment of the invention comprises: human cell culture medium as vehicle; and a phytocystatin recombinant polypeptide of rice (O. sativa) containing only the carboxy terminal portion (FIG. 1, part A)), having been administered to mammalian cells. Experiments with the administration of this embodiment of the invention composition in glioma cells have shown surprising and very promising results, as reported below.

In an experiment carried out with 4 different glioma lines, being 3 human lines and one murine line (C6) were plating in 24-well single-plate plates and treated the following day with the composition of the invention containing said polypeptide at concentrations of 1 uM, 10 uM and 50 uM. The treatment last 5 days with the presence of the composition of the invention and on the fifth day the medium was changed by one medium without the composition of the invention, the evaluation being done over time. Those skilled in the art will appreciate that the vehicle used in this embodiment of the invention can be replaced by other, according to the cell and/or organism to be treated and is well known in the art.

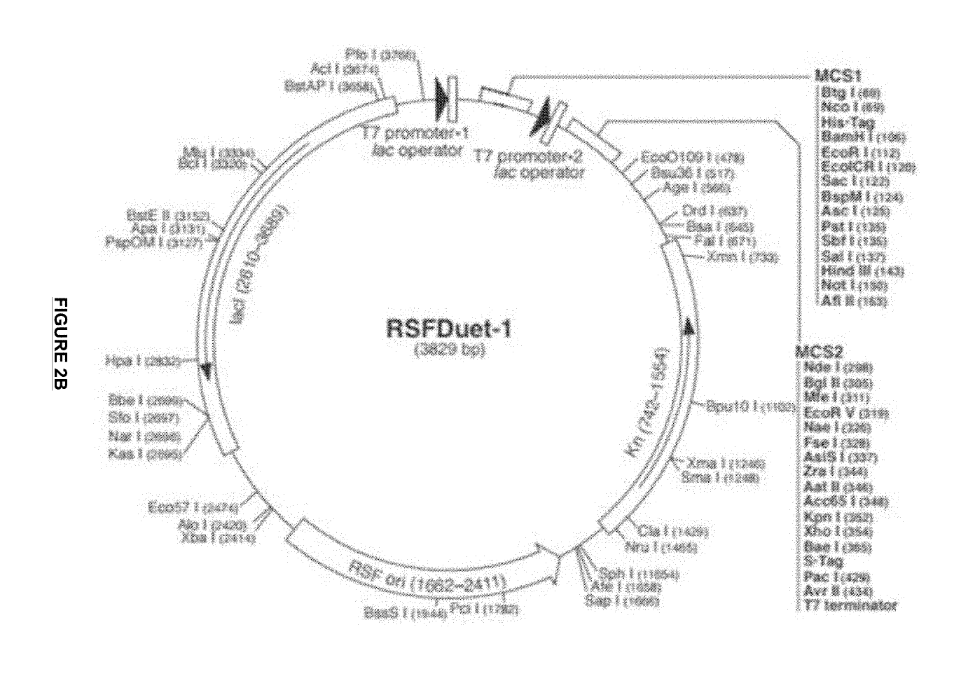

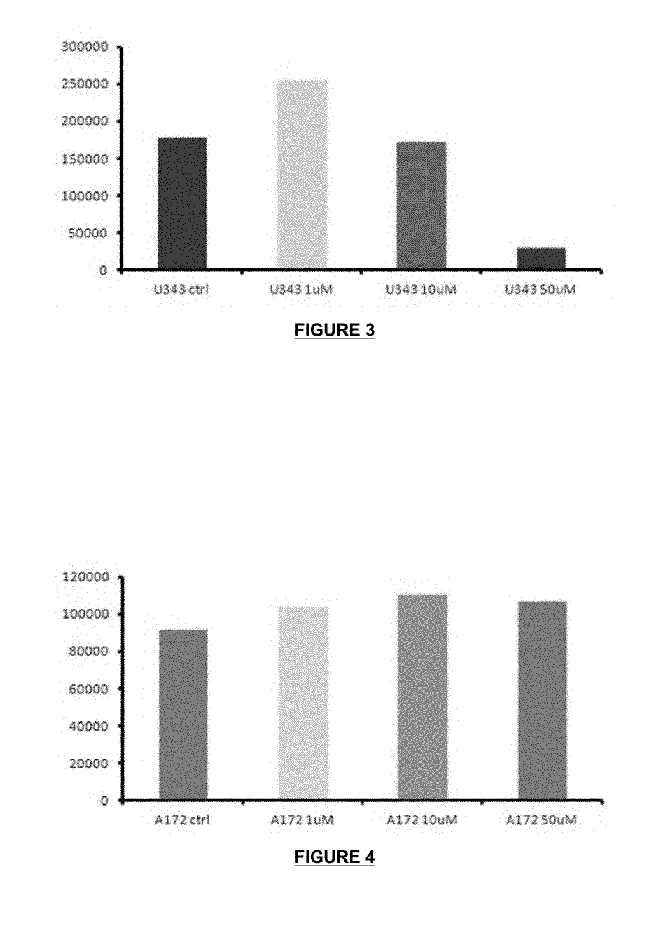

The results, shown in FIGS. 3-6, allow to conclude that: (i) under the conditions and cells tested, the 1 uM and 10 uM doses do not seem to have much long-term effect on these 4 lines tested; (ii) the dose of 50 uM gives evidence of an antitumor effect on glioma cells in the short term, although for some lines the effect is not maintained in the long term and the proliferative ability of the treated population appears to be restored; (iii) the 50 uM dose provided the complete elimination of one of the most resistant glioma lines, which are non-responsive to most of the available chemotherapeutic treatments.

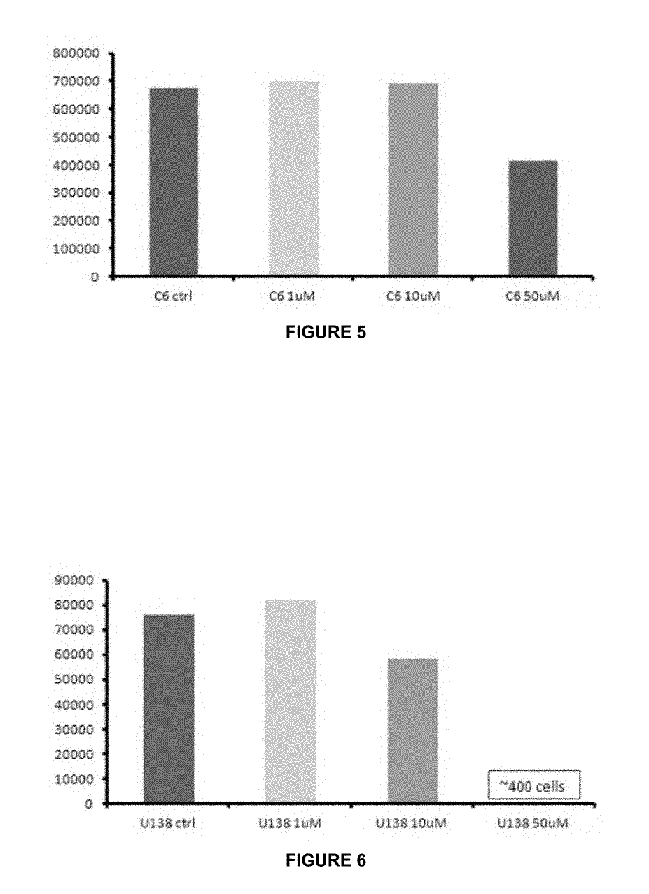

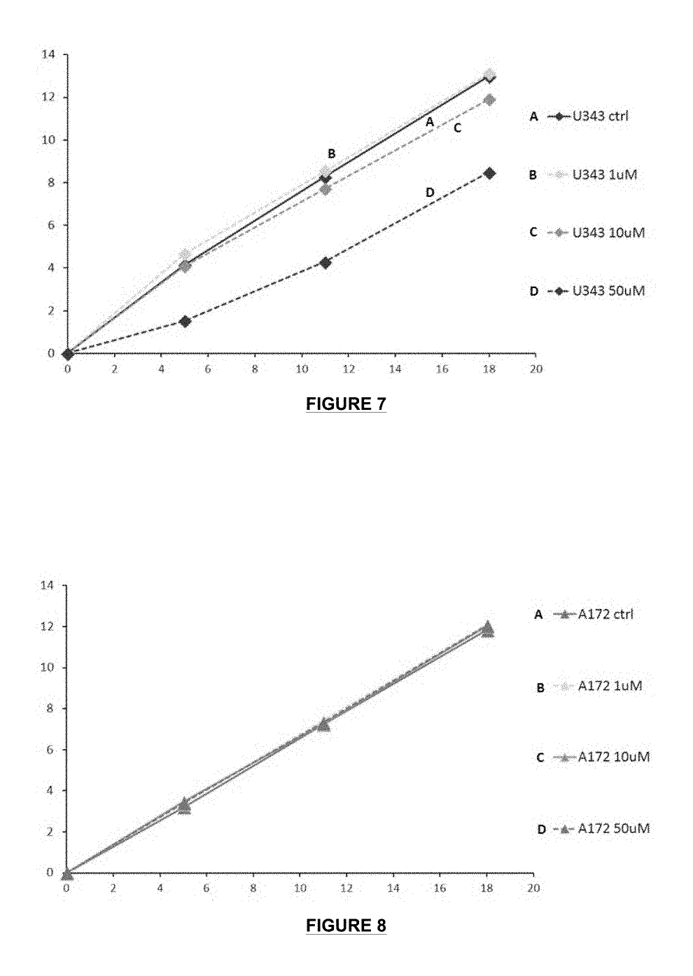

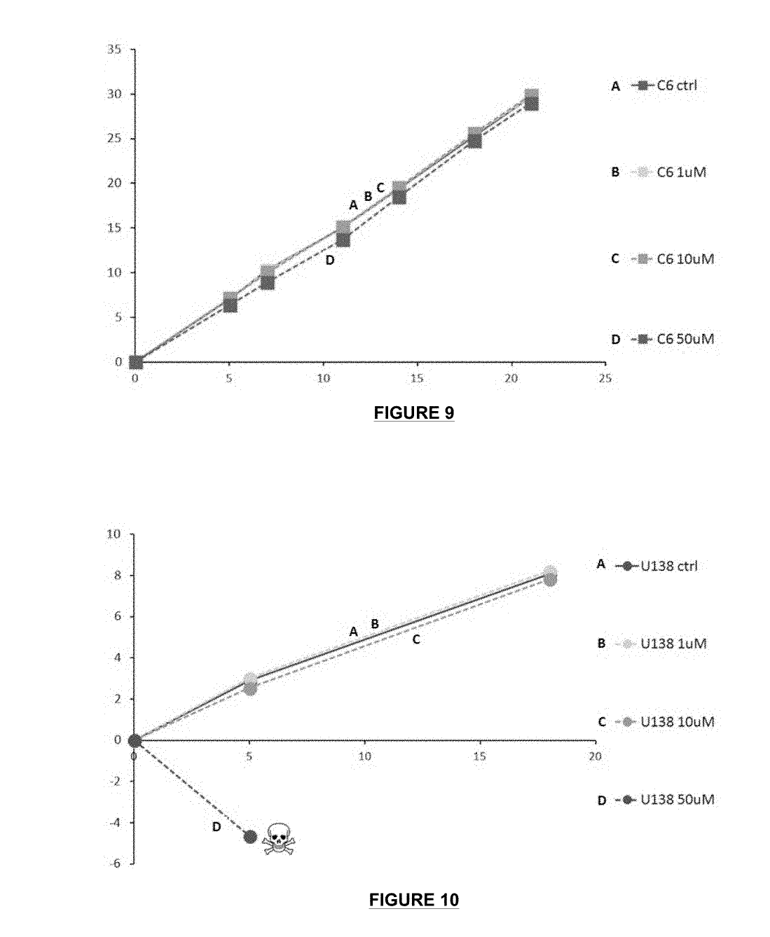

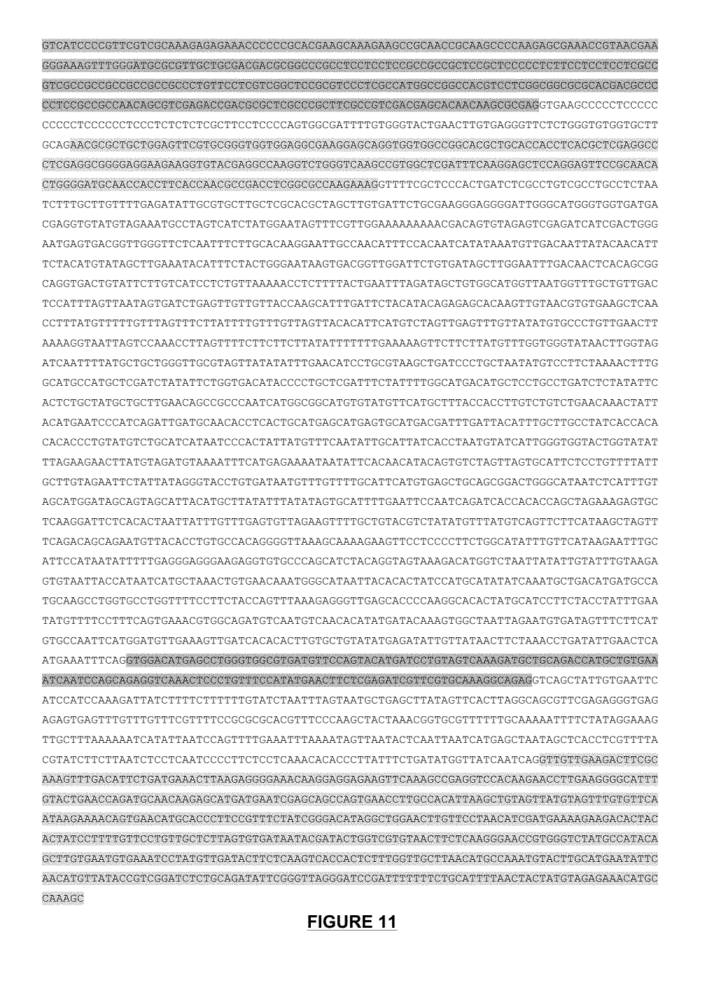

The graphs of FIGS. 7-10 illustrate the cell proliferation profiles of the lines treated with the composition of the invention, demonstrating promising effects. FIGS. 3-7 show the reduction of cell proliferation in cells U138. FIGS. 5 and 9 show the reduction of cell proliferation in cells C6. FIGS. 6 and 10 show the very substantial reduction of cell proliferation in cells U343, which are cells of glioma lines more chemotherapy resistant available in the art.

Those skilled in the art will appreciate the knowledge presented herein and may reproduce the invention in the embodiments presented and in other embodiments encompassed within the scope of the appended claims.

1. (canceled)

2. (canceled)

3. A pharmaceutical composition for cancer treatment of mammalian and/or other vertebrate comprising:
a pharmaceutically acceptable carrier; and
one or more polypeptides comprising one or more sequences having 70% or greater similarity to SEQ ID No: 6, or one or more sequences having 70% identity or greater with SEQ ID No: 6; or
one or more gene constructs useful for the expression in mammals and/or other vertebrates of one or more polypeptides as defined above.

4. The composition according to claim 3, wherein said polypeptide comprises an arrangement of two or more fused, repeated or sequenced polypeptide sequences of different legumain-selective ligand carboxy-terminal domains of mammalian and/or other vertebrate.

5. The composition according to claim 3, wherein said gene construct comprises an arrangement of two or more nucleotide sequences encoding said polypeptides fused in phase, repeated or in sequence of the same or different nucleotide sequences having equal or greater similarity to 70% with SEQ ID No: 5, or with identity equal to or greater identity to 70% with SEQ ID No: 5, for administration and expression of the polypeptide(s) directly into the mammalian/vertebrate organism.

6. The composition according to claim 3, wherein it is in the oral, sublingual, injectable, transdermal, topical, inhalable or suppository dosage form.

7. The composition according to claim 3, wherein it comprises a gene construct or expression vector comprising:
one or more nucleotide sequences having 70% or greater similarity to SEQ ID No: 5, or one or more nucleotide sequences having 70% identity or greater with SEQ ID No: 5;
a functional promoter in mammals and/or other vertebrates linked to the coding nucleotide sequence described above; and
a nucleotide sequence selected from: a transcription terminator nucleotide sequence; a selection marker; a secretion signal sequence; a sequence facilitating export; a nucleotide sequence encoding another protease inhibitory polypeptide sequence; combinations thereof, or still a plasmid comprising such sequences,
wherein any of the nucleotide sequences defined above is heterologous.

8. The composition according to claim 3, wherein said polypeptide is combined with one or more candidate molecules with legumain linker(s).

9. (canceled)

10. A method for the cancer treatment of mammals and/or other vertebrates comprising the use of a pharmaceutical composition comprising:
one or more polypeptides comprising one or more sequences having equal or greater than 70% of similarity to SEQ ID No: 6, or one or more sequences having an equal or greater than 70% of identity with SEQ ID No: 6; or
one or more gene constructs useful for the expression of one or more of said polypeptides,
wherein the mammals and/or other vertebrates are treated with a pharmaceutically effective amount of the pharmaceutical composition.

11. A process for the preparation of a pharmaceutical composition for cancer treatment of mammalian and/or other vertebrate cancer comprising a mixing step for mixing:
one or more polypeptides comprising one or more sequences having equal or greater than 70% similarity to SEQ ID No: 6, or one or more sequences having an identity equal or greater than 70% identity with SEQ ID No: 6; or
one or more gene constructs useful for the expression in mammals and/or other vertebrates of one or more of said polypeptides; and
a pharmaceutically acceptable carrier.

12. The composition according to claim 4, wherein it is in the oral, sublingual, injectable, transdermal, topical, inhalable or suppository dosage form.

13. The composition according to claim 5, wherein it is in the oral, sublingual, injectable, transdermal, topical, inhalable or suppository dosage form.


SEQUENCE LISTING (6 sequences)

SEQ ID NO: 1 - DNA, 3790 bp, Oryza sativa
  >LOC_Os01g16430 | Chr19325545..9329334 reverse
gtcatccccgttcgtcgcaaagagagaaaccccccgcacgaagcaaagaagccgcaaccgcaagccccaagagcgaaaccgtaacgaagggaaagtttgggatgcgcgttgctgcgacgacgcggcccgcctcctcctccgccgccgctccgctccccctcttcctcctcctcgccgtcgccgccgccgccgccgccctgttcctcgtcggctccgcgtccctcgccatggccggccacgtcctcggcggcgcgcacgacgccccctccgccgccaacagcgtcgagaccgacgcgctcgcccgcttcgccgtcgacgagcacaacaagcgcgaggtgaagccccctcccccccccctcccccctccctctctctcgcttcctccccagtggcgattttgtgggtactgaacttgtgagggttctctgggtgtggtgcttgcagaacgcgctgctggagttcgtgcgggtggtggaggcgaaggagcaggtggtggccggcacgctgcaccacctcacgctcgaggccctcgaggcggggaggaagaaggtgtacgaggccaaggtctgggtcaagccgtggctcgatttcaaggagctccaggagttccgcaacactggggatgcaaccaccttcaccaacgccgacctcggcgccaagaaaggttttcgctcccactgatctcgcctgtcgcctgcctctaatctttgcttgttttgagatattgcgtgcttgctcgcacgctagcttgtgattctgcgaagggaggggattgggcatgggtggtgatgacgaggtgtatgtagaaatgcctagtcatctatggaatagtttcgttggaaaaaaaaacgacagtgtagagtcgagatcatcgactgggaatgagtgacggttgggttctcaatttcttgcacaaggaattgccaacatttccacaatcatataaatgttgacaattatacaacatttctacatgtatagcttgaaatacatttctactgggaataagtgacggttggattctgtgatagcttggaatttgacaactcacagcggcaggtgactgtattcttgtcatcctctgttaaaaacctcttttactgaatttagatagctgtggcatggttaatggtttgctgttgactccatttagttaatagtgatctgagttgttgttaccaagcatttgattctacatacagagagcacaagttgtaacgtgtgaagctcaacctttatgtttttgtttagtttcttattttgtttgttagttacacattcatgtctagttgagtttgttatatgtgccctgttgaacttaaaaggtaattagtccaaaccttagttttcttcttcttatatttttttgaaaaagttcttcttatgtttggtgggtataacttggtagatcaattttatgctgctgggttgcgtagttatatatttgaacatcctgcgtaagctgatccctgctaatatgtccttctaaaactttggcatgccatgctcgatctatattctggtgacatacccctgctcgatttctattttggcatgacatgctcctgcctgatctctatattcactctgctatgctgcttgaacagccgcccaatcatggcggcatgtgtatgttcatgctttaccaccttgtctgtctgaacaaactattacatgaatcccatcagattgatgcaacacctcactgcatgagcatgagtgcatgacgatttgattacatttgcttgcctatcaccacacacaccctgtatgtctgcatcataatcccactattatgtttcaatattgcattatcacctaatgtatcattgggtggtactggtatatttagaagaacttatgtagatgtaaaatttcatgagaaaataatattcacaacatacagtgtctagttagtgcattctcctgttttattgcttgtagaattctattatagggtacctgtgataatgtttgttttgcattcatgtgagctgcagcggactgggcataatctcatttgtagcatggatagcagtagcattacatgcttatatttatatagtgcattttgaattccaatcagatcaccacaccagctagaaagagtgctcaaggattctcacactaattatttgtttgagtgttagaagttttgctgtacgtctatatgtttatgtcagttcttcataagctagtttcagacagcagaatgttacacctgtgccacaggggttaaagcaaaagaagttcctccccttctggcatatttgttcataagaatttgcattccataatatttttgagggagggaagaggtgtgcccagcatctacaggtagtaaagacatggtctaattatattgtatttgtaagagtgtaattaccataatcatgctaaactgtgaacaaatgggcataattacacactatccatgcatatatcaaatgctgacatgatgccatgcaagcctggtgcctggttttccttctaccagtttaaagagggttgagcaccccaaggcacactatgcatccttctacctatttgaatatgttttcctttcagtgaaacgtggcagatgtcaatgtcaacacatatgatacaaagtggctaattagaatgtgatagtttcttcatgtgccaattcatggatgttgaaagttgatcacacacttgtgctgtatatgagatattgttataacttctaaacctgatattgaactcaatgaaatttcaggtggacatgagcctgggtggcgtgatgttccagtacatgatcctgtagtcaaagatgctgcagaccatgctgtgaaatcaatccagcagaggtcaaactccctgtttccatatgaacttctcgagatcgttcgtgcaaaggcagaggtcagctattgtgaattcatccatccaaagattatcttttcttttttgtatctaatttagtaatgctgagcttatagttcacttaggcagcgttcgagagggtgagagagtgagtttgtttgtttcgttttccgcgcgcacgtttcccaagctactaaacggtgcgttttttgcaaaaattttctataggaaagttgctttaaaaaatcatattaatccagttttgaaatttaaaatagttaatactcaattaatcatgagctaatagctcacctcgttttacgtatcttcttaatctcctcaatccccttctcctcaaacacacccttatttctgatatggttatcaatcaggttgttgaagacttcgcaaagtttgacattctgatgaaacttaagaggggaaacaaggaggagaagttcaaagccgaggtccacaagaaccttgaaggggcatttgtactgaaccagatgcaacaagagcatgatgaatcgagcagccagtgaaccttgccacattaagctgtagttatgtagtttgtgttcaataagaaaacagtgaacatgcacccttccgtttctatcgggacataggctggaacttgttcctaacatcgatgaaaagaagacactacactatccttttgttcctgttgctcttagtgtgataatacgatactggtcgtgtaacttctcaagggaaccgtgggtctatgccatacagcttgtgaatgtgaaatcctatgttgatacttctcaagtcaccactctttggttgcttaacatgccaaatgtacttgcatgaatattcaacatgttataccgtcggatctctgcagatattcgggttagggatccgatttttttctgcattttaactactatgtagagaaacatgccaaagc

SEQ ID NO: 2 - DNA, 1252 bp, Artificial Sequence
  Complete nucleotide sequence of mature transcript equivalent to messenger RNA >LOC_Os01g16430.1
gtcatccccgttcgtcgcaaagagagaaaccccccgcacgaagcaaagaagccgcaaccgcaagccccaagagcgaaaccgtaacgaagggaaagtttgggatgcgcgttgctgcgacgacgcggcccgcctcctcctccgccgccgctccgctccccctcttcctcctcctcgccgtcgccgccgccgccgccgccctgttcctcgtcggctccgcgtccctcgccatggccggccacgtcctcggcggcgcgcacgacgccccctccgccgccaacagcgtcgagaccgacgcgctcgcccgcttcgccgtcgacgagcacaacaagcgcgagaacgcgctgctggagttcgtgcgggtggtggaggcgaaggagcaggtggtggccggcacgctgcaccacctcacgctcgaggccctcgaggcggggaggaagaaggtgtacgaggccaaggtctgggtcaagccgtggctcgatttcaaggagctccaggagttccgcaacactggggatgcaaccaccttcaccaacgccgacctcggcgccaagaaaggtggacatgagcctgggtggcgtgatgttccagtacatgatcctgtagtcaaagatgctgcagaccatgctgtgaaatcaatccagcagaggtcaaactccctgtttccatatgaacttctcgagatcgttcgtgcaaaggcagaggttgttgaagacttcgcaaagtttgacattctgatgaaacttaagaggggaaacaaggaggagaagttcaaagccgaggtccacaagaaccttgaaggggcatttgtactgaaccagatgcaacaagagcatgatgaatcgagcagccagtgaaccttgccacattaagctgtagttatgtagtttgtgttcaataagaaaacagtgaacatgcacccttccgtttctatcgggacataggctggaacttgttcctaacatcgatgaaaagaagacactacactatccttttgttcctgttgctcttagtgtgataatacgatactggtcgtgtaacttctcaagggaaccgtgggtctatgccatacagcttgtgaatgtgaaatcctatgttgatacttctcaagtcaccactctttggttgcttaacatgccaaatgtacttgcatgaatattcaacatgttataccgtcggatctctgcagatattcgggttagggatccgatttttttctgcattttaactactatgtagagaaacatgccaaagc

SEQ ID NO: 3 - DNA, 753 bp, Artificial Sequence
  Complete nucleotide sequence of the encoding region >LOC_Os01g16430.1 CDS
atgcgcgttgctgcgacgacgcggcccgcctcctcctccgccgccgctccgctccccctcttcctcctcctcgccgtcgccgccgccgccgccgccctgttcctcgtcggctccgcgtccctcgccatggccggccacgtcctcggcggcgcgcacgacgccccctccgccgccaacagcgtcgagaccgacgcgctcgcccgcttcgccgtcgacgagcacaacaagcgcgagaacgcgctgctggagttcgtgcgggtggtggaggcgaaggagcaggtggtggccggcacgctgcaccacctcacgctcgaggccctcgaggcggggaggaagaaggtgtacgaggccaaggtctgggtcaagccgtggctcgatttcaaggagctccaggagttccgcaacactggggatgcaaccaccttcaccaacgccgacctcggcgccaagaaaggtggacatgagcctgggtggcgtgatgttccagtacatgatcctgtagtcaaagatgctgcagaccatgctgtgaaatcaatccagcagaggtcaaactccctgtttccatatgaacttctcgagatcgttcgtgcaaaggcagaggttgttgaagacttcgcaaagtttgacattctgatgaaacttaagaggggaaacaaggaggagaagttcaaagccgaggtccacaagaaccttgaaggggcatttgtactgaaccagatgcaacaagagcatgatgaatcgagcagccagtga

SEQ ID NO: 4 - protein, 250 aa, Oryza sativa
  >LOC_Os01g16430.1
MRVAATTRPASSSAAAPLPLFLLLAVAAAAAALFLVGSASLAMAGHVLGGAHDAPSAANSVETDALARFAVDEHNKRENALLEFVRVVEAKEQVVAGTLHHLTLEALEAGRKKVYEAKVWVKPWLDFKELQEFRNTGDATTFTNADLGAKKGGHEPGWRDVPVHDPVVKDAADHAVKSIQQRSNSLFPYELLEIVRAKAEVVEDFAKFDILMKLKRGNKEEKFKAEVHKNLEGAFVLNQMQQEHDESSSQ

SEQ ID NO: 5 - DNA, 345 bp, Artificial Sequence
  Nucleotide sequence corresponding only to the extended carboxy-terminal region >LOC_Os01g16430.1 CDS
ggggatgcaaccaccttcaccaacgccgacctcggcgccaagaaaggtggacatgagcctgggtggcgtgatgttccagtacatgatcctgtagtcaaagatgctgcagaccatgctgtgaaatcaatccagcagaggtcaaactccctgtttccatatgaacttctcgagatcgttcgtgcaaaggcagaggttgttgaagacttcgcaaagtttgacattctgatgaaacttaagaggggaaacaaggaggagaagttcaaagccgaggtccacaagaaccttgaaggggcatttgtactgaaccagatgcaacaagagcatgatgaatcgagcagccagtga

SEQ ID NO: 6 - protein, 114 aa, Artificial Sequence
  Amino acid sequence corresponding only to the extended carboxy-terminal region >LOC_Os01g16430.1_carboxi
GDATTFTNADLGAKKGGHEPGWRDVPVHDPVVKDAADHAVKSIQQRSNSLFPYELLEIVRAKAEVVEDFAKFDILMKLKRGNKEEKFKAEVHKNLEGAFVLNQMQQEHDESSSQ